(19) United States
(12) Patent Application Publication
(10) Pub. No.: US 2018/0193767 A1
(43) Pub. Date: Jul. 12, 2018

(54) AMINES ANALYSIS BY ION CHROMATOGRAPHY

(71) Applicant: GENERAL ELECTRIC COMPANY, Schenectady, NY (US)

(72) Inventors: Timothy Mark SIVAVEC, Clifton Park, NY (US); Nathan MCLEAN, Glenville, NY (US)

(21) Appl. No.: 15/742,159

(22) PCT Filed: Jul. 5, 2016
(86) PCT No.: PCT/US2016/040939

§ 371 Date: Jan. 5, 2018

Related U.S. Application Data
(60) Provisional application No. 62/262,575, filed on Dec. 3, 2015.
(60) Provisional application No. 62/191,576, filed on Jul. 13, 2015.

(51) Int. Cl. B01D 15/16; B01D 15/36; G01N 30/34; G01N 30/96; G01N 30/30; G01N 30/46

(52) U.S. Cl. CPC B01D 15/161; B01D 15/166; B01D 15/362; G01N 2030/342; G01N 30/96; G01N 30/30; G01N 30/46; G01N 30/34

(57) ABSTRACT

Methods for separating, monitoring, identifying, and/or quantifying a plurality of ionic species in a mixture are disclosed herein. The ionic species can include a plurality of amines in an industrial fluid. The methods can include a first chromatography step, a second chromatography step, and optionally, a third chromatography step. The first chromatography step and second chromatography step can be performed simultaneously, for example in a dual channel apparatus, such that the method can be performed in less than about 24 hours. Disclosed herein are also methods for efficiently operating a refinery.

### FIELD OF THE DISCLOSURE

This disclosure relates generally to methods for monitoring ionic species in a mixture, particularly to monitoring ionic species in an industrial fluid.

### BACKGROUND OF THE DISCLOSURE

Salt fouling and associated corrosion in crude unit overhead systems are complex phenomena that impact refinery reliability, flexibility, and ultimately, profitability. Establishing an appropriate balance of physical, mechanical, and operational parameters, unique to each unit, is critical to minimizing fouling and corrosion throughout the crude unit. Factors such as amine chloride salt points, optimum accumulator pH, and overhead water ICP (initial condensation point, also referred to as water dew point) are interrelated and all affect the potential for system fouling and corrosion. There is a need for methods of monitoring, identifying, and/or quantifying ionic species in an industrial fluid.

Further, amines present in industrial fluids, for example in accumulator overhead water can come from a variety of sources (e.g., neutralizers, original crude source, upstream additives, hydrogen sulfide scavengers) and over time the mix of amines present changes. Therefore, there is a need for methods of monitoring, identifying, and/or quantifying a plurality of amines in an industrial fluid, wherein the methods are not affected by the presence of common interfering species (including cations, anions, crude oil). The methods disclosed herein address these and other needs.

### SUMMARY OF THE DISCLOSURE

Methods for analyzing a plurality of ionic species in a mixture are disclosed herein. In some embodiments, the methods can be used to separate, monitor, identify, and/or quantify a plurality of amines in a mixture. The method can include a first chromatography step, a second chromatography step, and optionally, a third chromatography step. The first chromatography step and second chromatography step can be performed simultaneously, for example in a dual channel apparatus. In some embodiments, the methods described herein can be performed in an amount of time of less than about 24 hours, less than about 10 hours, less than about 5 hours, or less than about 3 hours.

The first chromatography step can comprise introducing a sample of the mixture onto a first ion-exchange chromatography matrix, and eluting at least one first eluate from the first ion-exchange chromatography matrix under a first gradient eluting condition, using a first gradient eluent at a first elution temperature. The first gradient eluent can comprise an acid selected from citric acid, glycine, 2(N-morpholino)ethanesulfonic acid, methanesulfonic acid, acetic acid, formic acid, phosphoric acid, hydrochloric acid, nitric acid, sulfuric acid, phosphoric acid, oxalic acid, tartaric acid, benzoic acid, phthalic acid, and combinations thereof. In some embodiments, the first gradient eluting condition used in the methods can include an acid concentration gradient between about 1 mM to about 100 mM.

The first gradient eluent can further comprise a polar aprotic solvent such as acetonitrile. In some embodiments, the first gradient eluent used in the methods can include acetonitrile in an amount of from about 0.05% to about 25% by volume, based on the volume of the first gradient eluent. The first elution temperature can be from about 15° C. to about 45° C.

In some embodiments, the first gradient eluting condition includes a multistep gradient. In some examples, the first multistep gradient can include a first step, wherein the first gradient eluent comprises an acid concentration of from about 1 mM to about 10 mM; a second step, wherein the first gradient eluent comprises an acid concentration of from about 50 mM to about 100 mM; and a third step, wherein the first gradient eluent comprises an acid concentration of from about 1 mM to about 10 mM.

In some examples, the first multistep gradient can include a first step, wherein the first gradient eluent comprises an acid at a concentration of from about 2 mM to about 5 mM and acetonitrile in an amount of from about 0.05% to about 5% by volume, based on the total volume of the first gradient eluent. The first multistep gradient can further include a second step, wherein the first gradient eluent comprises an acid at a concentration of from about 25 mM to about 100 mM and acetonitrile in an amount of from about 5% to about 20% by volume, based on the total volume of the first gradient eluent. The first multistep gradient can also include a third step, wherein the first gradient eluent comprises an acid at a concentration of from about 2 mM to about 5 mM and acetonitrile in an amount of from about 0.05% to about 5% by volume, based on the volume of the first gradient eluent.

In some embodiments, the second chromatography step can comprise introducing a sample of the mixture onto a second ion-exchange chromatography matrix under a second gradient eluting condition, and eluting at least one second eluate from the second ion-exchange chromatography matrix, using a second gradient eluent at a second elution temperature. The second gradient eluent can comprise an acid as described herein. In some embodiments, the second gradient eluting condition used in the methods can include an acid concentration gradient between about 5 mM to about 100 mM.

In some embodiments, the second gradient eluting condition includes a multistep gradient. In some examples, the second multistep gradient condition can include a first step, wherein the second gradient eluent comprises an acid concentration of from about 3 mM to about 15 mM; a second step, wherein the second gradient eluent comprises an acid concentration of from about 50 mM to about 100 mM; and a third step, wherein the second gradient eluent comprises an acid concentration of from about 3 mM to about 10 mM.

In some embodiments, the second chromatography step can comprise introducing a sample of the mixture onto a second ion-exchange chromatography matrix under a first isocratic eluting condition, and eluting at least one second eluate from the second ion-exchange chromatography matrix, using a first isocratic eluent at a second elution temperature. In some embodiments, the first isocratic eluent used in the methods can include an acid concentration of from about 5 mM to about 20 mM or from about 8 mM to about 15 mM. The first isocratic eluent can comprise acetonitrile in an amount of from about 0.5% to about 5% or about 2% to about 4% by volume, based on the volume of the first isocratic eluent. The second elution temperature can be from about 15° C. to about 70° C.

The optional third chromatography step can comprise introducing a sample of the mixture onto a third ion-exchange chromatography matrix, and eluting at least one third eluate from the third ion-exchange chromatography matrix under a second isocratic condition, using a second isocratic eluent at a third elution temperature. The second isocratic eluent can comprise an acid as described herein, having a concentration of from about 5 mM to about 20 mM. The third elution temperature can be from about 35° C. to about 45° C.

The mixture of ionic species that can be analyzed using the methods described can include amines, metal ions, sulfides, sulfates, phosphates, nitrates, nitrites, halides, organic acids, perchlorates, selenates, cyanides, borates, and combinations thereof. In some embodiments, the mixture can include a plurality of cationic species. In some embodiments, the mixture can include a plurality of amines such as from about 5 to about 25 amines. In some examples, the mixture can include at least 5 amines, at least 10 amines, or at least 20 amines.

The method can include identifying and/or quantifying the separated ionic species using any suitable apparatus. Suitable apparatuses can include a mass spectrometer, a nuclear magnetic resonance spectrometer, a surface enhanced Raman spectrometer, a ultra-violet spectrophotometer, a fluorimeter, and combinations thereof. The minimum detection level of each ionic species in the mixture can be about 10 ppb.

The methods described herein are suitable for separating ionic species in an industrial fluid. Representative examples of industrial fluids can include a refinery fluid, a production fluid, cooling water, process water, drilling fluids, completion fluids, production fluids, crude oil, feed streams to desalting units, outflow from desalting units, refinery heat transfer fluids, gas scrubber fluids, refinery unit feed streams, refinery intermediate streams, finished product streams, and combinations thereof.

Disclosed herein are also methods for efficiently operating a refinery.

### BRIEF DESCRIPTION OF THE DRAWINGS

FIG. 1 is a graph showing an ion-exchange chromatogram of early eluting amines from a mixture.

FIG. 2 is a graph showing an ion-exchange chromatogram of mid eluting amines from a mixture.

FIG. 3 is a graph showing an ion-exchange chromatogram of late eluting amines from a mixture.

FIG. 4 is a graph showing an ion-exchange chromatogram of early eluting amines from a mixture.

FIG. 5 is a graph showing an ion-exchange chromatogram of late eluting amines from a mixture.

### DETAILED DESCRIPTION

Disclosed herein are methods for monitoring, separating, identifying, and/or quantifying a plurality of ionic species in a mixture. In some aspects, the methods provide for analysis of a plurality of amines in a mixture. The method can include a multi-step ion-exchange chromatography method for separating and/or monitoring the ionic species in the mixture. In some embodiments, the method can include two or more ion-exchange chromatography steps. The ionic species can be identified and/or quantified, following separation, using any suitable method known in the art.

“Ion-exchange chromatography” or “ion chromatography” as used herein, refers to a chromatographic process in which an ionizable solute(s) of interest (e.g., an amine) interacts with an oppositely charged ligand linked (e.g., by covalent attachment) to a solid phase ion-exchange material under appropriate conditions of pH, temperature, eluent, and/or conductivity, such that the solute(s) of interest interacts non-specifically with the charged ligand as a function of their net surface charge. As a result, the solute(s) of interest separates according to differences in their net charge. Ion-exchange chromatography includes cation-exchange chromatography, anion-exchange chromatography, and mixed mode chromatography.

The methods described herein can be carried out using commercially available ion-exchange chromatography systems. Suitable ion-exchange chromatography systems include, but are not limited to, ICS-5000 single channel or dual channel apparatuses supplied by Dionex™ or a 940 Professional IC Vario apparatus available from Metrohm™. The ion-exchange can be carried out using commercially available columns known in the art and used in ion chromatography. In some embodiments, a cation-exchange column comprising a chromatography matrix packed with an acidic cation exchanger can be used. In some embodiments, the chromatography matrix can comprise carboxylic acid groups, phosphonic acid groups, sulfonic acid groups, or combinations thereof. Suitable examples of commercially available cation-exchange columns include, but are not limited to, CS11, CS12, CS14, CS15, CS16, CS17, CS18, and CS19 supplied under the tradename IonPac by Dionex™; TSKgel IC-Cation or TSKgel IC-Cation I/II HR supplied by TOSOH Corporation; Shodex YK-421 supplied by Showa Denko K.K; and C6 supplied by Metrohm™ under the tradename Metrosep™.

The eluent (also referred to herein as an “elution buffer”) can include any suitable eluent known in the art for eluting an ionic species from an ion-exchange chromatography matrix. Suitable eluents can include citric acid, glycine, 2(N-morpholino) ethanesulfonic acid, methanesulfonic acid, acetic acid, formic acid, phosphoric acid, hydrochloric acid, nitric acid, sulfuric acid, phosphoric acid, oxalic acid, tartaric acid, benzoic acid, phthalic acid, and combinations thereof. The methods described herein can include gradient and/or isocratic flow of the eluent.

The eluent can further include a polar aprotic solvent. Suitable examples of polar aprotic solvents include solvents comprising an amide, ketone, nitrile, sulfoxide, sulfone, or alkylene carbonate. In some examples, the polar aprotic solvent can be selected from acetonitrile, tetrahydrofuran, dimethylacetamide, methyl ethyl ketone, butyronitrile, dimethyl sulfoxide, sulfolane, propylene carbonate, and butylene carbonate.

The methods for separating the ionic species can be carried out at any suitable temperature for improved selectivity of ion-exchange reactions and efficiency of the ion-exchange column, thus influencing the quality of the separation. In particular, changes in the temperature of a column can cause changes in the thermodynamic functions (free energy, enthalpy, and entropy) of the column that results in increased or decreased retention of the species depending on, for example, the acidity of cation-exchange functional groups and the eluent used. In some embodiments, the elution temperature, (including the temperature of the column(s) including the eluent and the chromatography matrix) can be from about 10° C. to about 80° C. For example, the elution temperature can be from about 15° C. to about 70° C., about 25° C. to about 70° C., about 30° C. to about 70° C., about 35° C. to about 70° C., about 15° C. to about 45° C., or about 15° C. to about 35° C.

The methods described herein are suitable for monitoring, separating, identifying, and/or quantifying a plurality of cationic or anionic species in a mixture. Suitable ionic species can include amines, metal ions, sulfides, sulfates, phosphates, nitrates, nitrites, halides, organic acids, perchlorates, selenates, cyanides, borates, and combinations thereof. Representative examples of ionic species can include primary, secondary, tertiary, and quarternary amines such as methylamine, ethylamine, ethanolamine, cyclohexylamine, morpholine, monoethanolamine, dimethylethanolamine, and pyridines; ammonia; alkali and alkaline metals such as sodium, potassium, magnesium, and calcium; and combinations thereof.

In some embodiments, the methods provide for separation and/or quantification of a plurality of amines in a mixture. In some examples, the mixture can comprise at least 5 amines, at least 10 amines, or at least 20 amines. In some embodiments, the mixture can comprise from about 5 to about 25 amines.

The mixture can be an industrial fluid, including liquids and gases. Industrial fluids also include materials that may be solid at ambient temperatures but are liquid during an industrial process. The industrial fluid can include aqueous and non-aqueous fluids, including emulsions and other multiphase fluids which are admixtures of aqueous and non-aqueous fluids and which are present in the exploration for or production of oil and gas, during the refining of crude oil, and during the production of chemical products. In some embodiments, the industrial fluid can include a refinery fluid, a production fluid, cooling water, process water, oil field drilling and completion fluids, oil and gas well production fluids, crude oil, feed streams to desalting units, outflow from desalting units, refinery and chemical plant heat transfer fluids, gas scrubber fluids, chemical plant and refinery unit feed streams, refinery and chemical plant intermediate streams, and refinery and chemical plant production and finished product streams, and combinations thereof.

The industrial fluid or an industrial fluid stream can be introduced directly into the ion-exchange chromatography apparatus. In some embodiments, the mixture can be pre-treated prior to ion-exchange chromatography. For example, particulate matter can be removed from the sample by filtration prior to introducing the sample into the ion-exchange chromatography apparatus. In some embodiments, the pH of the mixture can be altered to for example, improve separation or increase interaction between the ionic species and the chromatography matrix, depending on the nature of the ionic species. In some embodiments, an industrial fluid can be treated with a pre-concentrator to increase the relative concentration of an ionic species of interest, reducing the presence of an undesirable fluid. In some embodiments, the industrial fluid can be subjected to a chemical treatment or derivatization. In some embodiments, the industrial fluid can be subjected to an extraction process or heating prior to being introduced into the ion-exchange chromatography apparatus.

The methods for separating the ionic species can include a first chromatography step, a second chromatography step, and optionally, a third chromatography step. Each chromatography step can be particularly carried out under conditions such that separation of the respective ions in the separation column is optimized. Such conditions can include, for example, the chromatographic matrix, flow rates of the eluent, the chemistry of the eluent, temperature, and the concentration of the sample.

For each chromatography step, at least one eluate can be obtained. “Eluate” as used herein, refers to a combination of the eluent and ionic species exiting the chromatography matrix. As discussed herein, the various compositions of the ionic species travel at different speeds allowing separation of the ionic species. In some embodiments, the eluate can be collected as one or more fractions, using any suitable method known in the art, to obtain one or more pure or substantially pure eluates for further analysis. “Substantially pure” as used herein, refers to an eluate, for example, with greater than 95 wt % of the desired ionic species, based on the total weight of solute in the eluate. In some embodiments, the eluate contains low amounts of undesirable species and can include less than 5 wt %, less than 3 wt %, less than 2 wt %, less than 1 wt %, or less than 0.5 wt % undesirable species, based on the total weight of solute in the eluate.

The first chromatography step can include introducing a sample of the mixture onto a first ion-exchange chromatography matrix. The first ion-exchange chromatography matrix can include a carboxylate-functionalized resin. In some aspects, where the method is used for separating amine salts, the first ion-exchange chromatography matrix can be suitable for separating adjacent eluting cations including metal ions such as sodium, potassium, and calcium; ammonium; and/or short-chained amines, including alkylamines and alkanolamines. In some embodiments, a high capacity column that provides high loadability and resolution, while also suitable for disparate concentrations of species in a variety of sample matrices can be used as the first ion-exchange chromatography matrix. In some examples, an IonPac™ CS14, IonPac™ CS15, IonPac™ CS16, or Metrohm™ Metrosep C6 column can be used as the first chromatography matrix.

The first chromatography step can include eluting at least one early eluting eluate (also referred to herein as “first eluate”) from the first ion-exchange chromatography matrix under a first gradient eluting condition. The first gradient eluting condition can include using a first gradient eluent at a first elution temperature. In some embodiments, a plurality of early eluting eluate fractions, comprising separated or substantially separated ionic species, can be obtained.

The first gradient eluent can include any one of the eluents described herein. In some embodiments, the first gradient eluent can comprise an acid, for example, methanesulfonic acid or oxalic acid. The first gradient eluent can have a pH of less than about 7, for example, about pH 6 or less, about pH 5 or less, about pH 4 or less, about pH 3 or less, or about pH 2 or less. The concentration of acid in the first gradient eluent can be from about 0.5 mM to about 100 mM.

In some aspects, the first gradient eluting condition uses an acid concentration gradient (i.e., the change in concentration of the acid in the eluent over time), encompassing any gradient from about 0.5 mM to about 100 mM. For example, the acid concentration gradient can be from about 1 mM to about 100 mM, about 1 mM to about 80 mM, or about 3 mM to about 70 mM. In some embodiments, the first gradient eluting condition includes a multistep gradient (or a “first multistep gradient”). The first multistep gradient can include two or more steps. For example, the first multistep gradient can include a first step, a second step, and optionally a third step.

In some embodiments, the first multistep gradient can include a first step, wherein the first gradient eluent comprises an acid concentration of from about 1 mM to about 10 mM; a second step, wherein the first gradient eluent comprises an acid concentration of from about 50 mM to about 100 mM; and a third step, wherein the first gradient eluent comprises an acid concentration of from about 1 mM to about 10 mM.

The first gradient eluent can further comprise a polar aprotic solvent, for example, acetonitrile. The polar aprotic solvent in the first gradient eluent can be in an amount of from about 0.05% to about 25% by volume, based on the total volume of the first gradient eluent. In some embodiments, the polar aprotic solvent in the first gradient eluent can be in an amount of about 0.05% or greater, about 0.1% or greater, about 0.2% or greater, about 0.25% or greater, about 0.5% or greater, about 1% or greater, about 2% or greater, about 3% or greater, about 5% or greater, about 6% or greater, about 7% or greater, about 8% or greater, about 9% or greater, about 10% or greater, about 11% or greater, about 12% or greater, about 15% or greater, about 18% or greater, about 20% or greater, about 22% or greater, or about 25% or greater by volume, based on the volume of the first gradient eluent. In some embodiments, the polar aprotic solvent in the first gradient eluent can be in an amount of about 30% or less, about 25% or less, about 20% or less, or about 15% or less by volume, based on the volume of the first gradient eluent. In some embodiments, the polar aprotic solvent in the first gradient eluent can be in an amount of about 0.1% to about 25%, about 0.1% to about 20%, or about 0.1% to about 15% by volume, based on the volume of the first gradient eluent. In some examples, the first gradient eluent does not include a polar aprotic solvent. In some examples, the first gradient eluent does not include acetonitrile.

In some embodiments, the first multistep gradient can include a first step, wherein the concentration of acid in the first gradient eluent is from about 2 mM to about 5 mM or from about 3 mM to about 4 mM. The polar aprotic solvent, such as acetonitrile, can be in amount of from about 0.05% to about 5% or about 0.1% to about 1% by volume, based on the volume of the first gradient eluent. In some embodiments, the first multistep gradient can include a second step, wherein the concentration of acid in the first gradient eluent is from about 25 mM to about 100 mM or from about 40 mM to about 60 mM. The polar aprotic solvent, such as acetonitrile, can be in amount of from about 5% to about 25% or about 5% to about 15% by volume, based on the volume of the first gradient eluent. In some embodiments, the first multistep gradient can include a third step, wherein the concentration of acid in the first gradient eluent is from about 2 mM to about 5 mM or from about 3 mM to about 4 mM. The polar aprotic solvent, such as acetonitrile, can be in amount of from about 0.05% to about 5% or about 0.1% to about 1% by volume, based on the volume of the first gradient eluent.

The first elution temperature can be about 15° C. or greater. In some embodiments, the first elution temperature can be from about 15° C. to about 45° C., about 20° C. to about 45° C., about 25° C. to about 45° C., about 20° C. to about 40° C., about 37° C. to about 45° C., about 37° C. to about 43° C., or about 38° C. to about 42° C. In some examples, the first elution temperature can be about 20° C., about 22° C., about 24° C., about 25° C., about 26° C., about 28° C., about 30° C., about 32° C., about 34° C., about 35° C., about 36° C., about 37° C., about 38° C., about 39° C., about 40° C., about 41° C., about 42° C., about 43° C., about 44° C., or about 45° C.

The first chromatography step can be performed within about 120 minutes or less. In some embodiments, the first chromatography step can be performed from about 5 minutes to about 100 minutes, about 10 minutes to about 90 minutes, about 10 minutes to about 60 minutes, about 20 minutes to about 60 minutes, about 20 minutes to about 50 minutes, or about 20 minutes to about 40 minutes. For example, the first chromatography step can be performed within about 110 minutes or less, about 100 minutes or less, about 90 minutes or less, about 80 minutes or less, about 75 minutes or less, about 70 minutes or less, about 60 minutes or less, or about 50 minutes or less.

The second chromatography step can include introducing a sample of the mixture onto a second ion-exchange chromatography matrix. The second ion-exchange chromatography matrix can include a carboxylate-functionalized resin. In some embodiments, where the method is used for separating amine salts, the second chromatography matrix can be the same as the first chromatography matrix. For example, an IonPac™ CS14, IonPac™ CS15, IonPac™ CS16, or Metrohm™ Metrosep C6 column can be used as the second chromatography matrix.

The second chromatography step can be performed sequential to or simultaneously with the first chromatography step. In some embodiments, the second chromatography step is performed simultaneously with the first chromatography step. For example, a dual channel ion-exchange chromatography system can be used, wherein the mixture can be injected via a single injection (i.e., one injection into two different eluent streams) or two injections (one injection per eluent stream). As such, the first channel can carry out the first chromatography step and the second channel carry out the second chromatography step, simultaneously. In some embodiments, the second chromatography step is performed sequential to the first chromatography step.

In some embodiments, the second chromatography step can include eluting at least one mid-eluting eluate (also referred to herein as “second eluate”) from the second ion-exchange chromatography matrix under a second gradient eluting condition. The second gradient eluting condition can include using a second gradient eluent at a second elution temperature. In some embodiments, a plurality of mid-eluting eluate fractions, comprising separated or substantially separated ionic species, can be obtained.

The second gradient eluent can include any one of the eluent described herein. In some embodiments, the second gradient eluent can comprise an acid, for example, methanesulfonic acid or oxalic acid. The second gradient eluent can have a pH of less than about 7, for example, about pH 6 or less, about pH 5 or less, about pH 4 or less, about pH 3 or less, or about pH 2 or less. The concentration of acid in the second gradient eluent can be from about 5 mM to about 100 mM.

In some embodiments, the second gradient eluting condition uses an acid concentration gradient encompassing any gradient from about 5 mM to about 100 mM. For example, the acid concentration gradient can be from about 5 mM to about 90 mM, about 5 mM to about 80 mM, about 5 mM to about 70 mM, or about 8 mM to about 70 mM. In some embodiments, the second gradient eluting condition includes a multistep gradient (or a “second multistep gradient”). The second multistep gradient can include two or more steps. For example, the second multistep gradient can include a first step, a second step, and optionally a third step.

In some embodiments, the second multistep gradient can include a first step, wherein the second gradient eluent comprises an acid concentration of from about 3 mM to about 20 mM or from about 3 mM to about 10 mM; a second step, wherein the second gradient eluent comprises an acid concentration of from about 25 mM to about 100 mM or from about 40 mM to about 60 mM; and a third step, wherein the second gradient eluent comprises an acid concentration of from about 1 mM to about 10 mM or from about 1 mM to about 5 mM.

The second gradient eluent can further comprise a polar aprotic solvent as described herein. In some examples, the second gradient eluent does not include a polar aprotic solvent. In some examples, the second gradient eluent does not include acetonitrile.

In some embodiments, the second chromatography step can include eluting at least one eluate from the second ion-exchange chromatography matrix under a first isocratic eluting condition. The first isocratic eluting condition includes using a first isocratic eluent at a second elution temperature. The first isocratic eluent can include any one of the eluent described herein. In some embodiments, the first isocratic eluent can comprise an acid, for example, methanesulfonic acid or oxalic acid. The first isocratic eluent can have a pH of less than about 7, for example, about pH 6 or less, about pH 5 or less, about pH 4 or less, about pH 3 or less, or about pH 2 or less. The concentration of acid in the first isocratic eluent can be from about 5 mM to about 20 mM or from about 5 mM to about 15 mM.

The first isocratic eluent can further comprise a polar aprotic solvent, for example, acetonitrile. The polar aprotic solvent in the first isocratic eluent can be in an amount of from about 0.5% to about 5% by volume, based on the total volume of the first isocratic eluent. In some embodiments, the polar aprotic solvent in the first isocratic eluent can be in an amount of about 0.5% or greater, about 1% or greater, about 1.5% or greater, about 2% or greater, about 2.5% or greater, about 3% or greater, about 4% or greater, or about 5% or greater by volume, based on the volume of the first isocratic eluent. In some embodiments, the polar aprotic solvent in the first isocratic eluent can be in an amount of about 5% or less, about 4% or less, or about 3% or less by volume, based on the volume of the first isocratic eluent. In some embodiments, the polar aprotic solvent in the first isocratic eluent can be in an amount of about 1% to about 5% or about 2% to about 5% by volume, based on the volume of the first isocratic eluent.

The second elution temperature can be about 15° C. or greater. In some embodiments, the second elution temperature can be from about 15° C. to about 70° C., 15° C. to about 65° C., 15° C. to about 50° C., 15° C. to about 30° C., 20° C. to about 30° C., 60° C. to about 70° C., about 62° C. to about 67° C., or about 63° C. to about 67° C. In some examples, the second elution temperature can be about 15° C., about 20° C., about 25° C., about 30° C., about 35° C., about 40° C., about 45° C., about 50° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C.

The second chromatography step can be performed within about 100 minutes or less. In some embodiments, the second chromatography step can be performed from about 5 minutes to about 100 minutes, about 10 minutes to about 90 minutes, about 10 minutes to about 60 minutes, about 20 minutes to about 60 minutes, or about 20 minutes to about 50 minutes. For example, the second chromatography step can be performed within about 90 minutes or less, about 80 minutes or less, about 75 minutes or less, about 70 minutes or less, about 60 minutes or less, or about 50 minutes or less.

Optionally, the methods described herein can include a third chromatography step. The third chromatography step can include introducing a sample of the mixture onto a third ion-exchange chromatography matrix. The third ion-exchange chromatography matrix can include a carboxylate-functionalized resin. In some embodiments, where the method is used for separating amine salts, the third chromatography matrix can be suitable for separating polar amines and moderately hydrophobic and polyvalent amines. In some embodiments, a high capacity column that provides for acidic gradient separation can be used as the third ion-exchange chromatography matrix. In some examples, an IonPac™ CS17, IonPac™ CS18, or IonPac™ CS19 column can be used as the third chromatography matrix. The third chromatography step can be performed sequential to the second chromatography step.

The third chromatography step can include eluting at least one late eluting eluate (also referred to herein as “third eluate”) from the third ion-exchange chromatography matrix under a second isocratic eluting condition. The second isocratic eluting condition includes using a second isocratic eluent at a third elution temperature. In some embodiments, a plurality of late eluting eluate fractions, comprising separated or substantially separated ionic species, can be obtained.

The second isocratic eluent can include any one of the eluent described herein. In some embodiments, the second isocratic eluent can comprise an acid, for example, methanesulfonic acid or oxalic acid. The second isocratic eluent can have a pH of less than about 7, for example, about pH 6 or less, about pH 5 or less, about pH 4 or less, about pH 3 or less, or about pH 2 or less. The concentration of acid in the second isocratic eluent can be about 5 mM or greater. In some embodiments, the concentration of acid in the second isocratic eluent can be from about 5 mM to about 20 mM, about 5 mM to about 15 mM, or about 10 mM.

The third elution temperature can be from about 35° C. to about 45° C. In some embodiments, the third elution temperature can be from about 37° C. to about 45° C., about 37° C. to about 43° C., or about 38° C. to about 42° C. In some examples, the third elution temperature can be about 36° C., about 37° C., about 38° C., about 39° C., about 40° C., about 41° C., about 42° C., about 43° C., about 44° C., or about 45° C.

The third chromatography step can be performed within about 60 minutes or less. In some embodiments, the third chromatography step can be performed from about 5 minutes to about 60 minutes, about 10 minutes to about 60 minutes, about 10 minutes to about 50 minutes, or about 20 minutes to about 60 minutes. For example, the third chromatography step can be performed within about 50 minutes or less, or about 45 minutes or less.

In some embodiments, the method for separating and/or monitoring a plurality of ionic species such as a plurality of amines in a mixture can include (a) a first chromatography step comprising (i) introducing a sample of the mixture onto a first ion-exchange chromatography matrix; and (ii) eluting at least one first eluate from the first ion-exchange chromatography matrix under a first multistep gradient eluting condition, using a first gradient eluent at a first elution temperature; and (b) a second chromatography step comprising (i) introducing a sample of the mixture onto a second ion-exchange chromatography matrix; and (ii) eluting at least one second eluate from the second ion-exchange chromatography matrix under a first isocratic condition, using a first isocratic eluent at a second elution temperature. In some embodiments, the mixture comprises at least five amines and the method can be performed in less than about 3 hours.

In some embodiments, the method for separating and/or monitoring a plurality of ionic species such as a plurality of amines in a mixture can include (a) a first chromatography step comprising (i) introducing a sample of the mixture onto a first ion-exchange chromatography matrix; and (ii) eluting at least one first eluate from the first ion-exchange chromatography matrix under a first gradient eluting condition, using a first gradient eluent at a first elution temperature; (b) a second chromatography step comprising (i) introducing a sample of the mixture onto a second ion-exchange chromatography matrix; and (ii) eluting at least one second eluate from the second ion-exchange chromatography matrix under a second gradient eluting condition, using a second gradient eluent at a second elution temperature; and (c) a third chromatography step comprising (i) introducing a sample of the mixture onto a third ion-exchange chromatography matrix; and (ii) eluting at least one third eluate from the third ion-exchange chromatography matrix under a second isocratic eluting condition, using a second isocratic eluent at a third elution temperature. In some embodiments, the mixture comprises at least five amines and the method can be performed in less than about 24 hours.

The eluates (including the at least one first eluate, the at least one second eluate, and the at least one third eluate) can be identified and/or quantified using any suitable apparatus know in the art and used to identify and/or quantify ionic species. In some examples, apparatuses for mass spectrometry, nuclear magnetic resonance, surface enhanced Raman scattering, ultra-violet spectrophotometry, fluorescence, conductivity, and combinations thereof can be used for identifying and/or quantifying the ionic species of interest. In some embodiments, a hybrid apparatus incorporating the ion-exchange chromatography system can be used in the methods described herein, for monitoring, separating, identifying, and/or quantifying the ionic species in a mixture. For example, an ICS-5000+TSQ Quantum Access Max triple quad or an ICS-5000+CD Conductivity Detector, supplied by Thermo Scientific′ and Dionex™ can be used in the methods described herein.

The minimum detection level of each ionic species in the mixture can be about 10 ppb or greater. In some embodiments, the detection level of each ionic species in the mixture can be about 1 ppm or greater, about 2 ppm or greater, about 3 ppm or greater, about 4 ppm or greater, about 4 ppm or greater, or about 10 ppm or greater.

The methods described herein can be performed and provide results in a short time. As a result, the methods can be employed in automated process control applications. A “short time,” as used herein generally refers to “sufficiently fast enough to be employed in controlling an industrial process” and specifically less than about 24 hours. In some embodiments, the method can be performed and produce results in less than about 10 hours, less than about 8 hours, less than about 6 hours, less than about 5 hours, less than about 4 hours, or less than about 3 hours.

Described herein are also methods for controlling an industrial device or an industrial process using the results of the output from the ion-exchange apparatus and/or the apparatus for identifying and/or quantifying the ionic species in the mixture. In some embodiments, the output may be employed directly to control a parameter of a process. Parameters related to, for example an industrial fluid that may be altered based on the results or data obtained related to the identified ionic species and the respective amount of the identified ionic species within the industrial fluid can include temperature, amount of the composition, pressure, and combinations thereof. For example, the temperature of a process may be altered in order to avoid the formation or deposition of solid amine hydrochloride salts within the process equipment if the concentration of a particular amine is determined to be above a pre-determined threshold value. As another example, the amount of specific amines or inorganic ions (such as chlorides) may be used to optimize process parameters of the desalter. The parameter may be altered upstream or downstream of the location of the analyzed sample. For example, contaminant removal technology may be applied at the desalter (upstream) based on the quantitation of amines in a water sample from the overhead system of the atmospheric distillation tower (downstream).

The output from the ion-exchange apparatus and/or the apparatus for identifying and/or quantifying the ionic species can also be used to speed up or slow down a specific process stream in response to the concentration of the undesirable compound. In another example, the output can be used to change the pH of a process stream, optimize the dosage of additives such as corrosion inhibitors, hydrate inhibitors, anti-fouling agents, antifoaming agents, anti-scaling agents, demulsifiers, and the like. In another example, the output from the ion-exchange apparatus and/or the apparatus for identifying and/or quantifying the ionic species can be employed as input into a computer model of a process. This may be used to indicate changes to feed stream rates, temperature, and/or pressures for efficient operation of a refinery.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope of the disclosure. Unless indicated otherwise, parts are parts by weight, temperature is in ° C. or is at ambient temperature, and pressure is at or near atmospheric.

### Example 1: Analysis of an Amine Mixture

### Material:

Sample Containing a Mixture of Amines

### First Chromatography Step—Chromatography Conditions:

### Dionex ICS3000

IC column: CS16 plus CG16 guard column

Column Temperature: 39° C.

Injection Volume: 10 uL

Eluent: Methanesulfonic acid (MSA)

Eluent Flow Rate: 0.5 ml/min

Separation Parameters: 3 mM MSA isocratic to 100 min, step to 70 mM MSA at 100 min, isocratic at 70 mM MSA to 110 min, step to 3 mM MSA at 110 min, isocratic at 3 mM to 120 min

Detector: Conductivity

Detector temperature: 20° C.

Run time: 120 min

### Results:

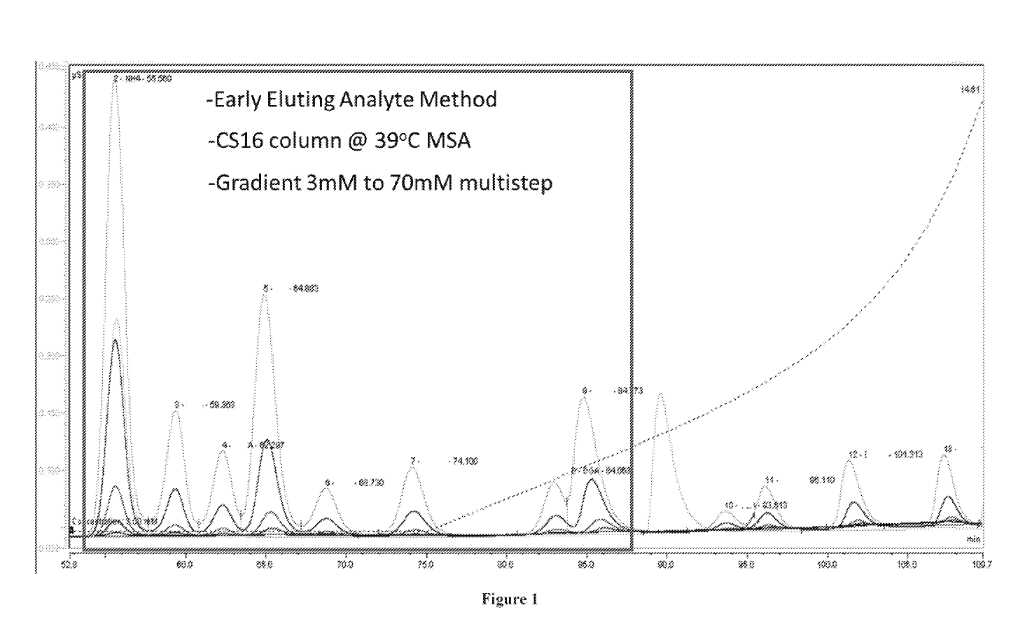

The result of the analysis is presented in FIG. 1.

### Second Chromatography Step—Chromatography Conditions:

### Dionex ICS3000

IC column: CS16 plus CG16 guard column

Column Temperature: 65° C.

Injection Volume: 10 uL

Eluent: Methanesulfonic Acid (MSA)

Eluent Flow Rate: 0.5 ml/min

Separation Parameters: 5 mM MSA isocratic to 35 min, gradient to 9 mM MSA at 90 min, step to 70 mM MSA at 90 min, isocratic at 70 mM to 105 min, step to 5 mM at 105 min, isocratic at 5 mM to 120 min

Detector: Conductivity

Detector temperature: 20° C.

Run time: 120 min

### Results:

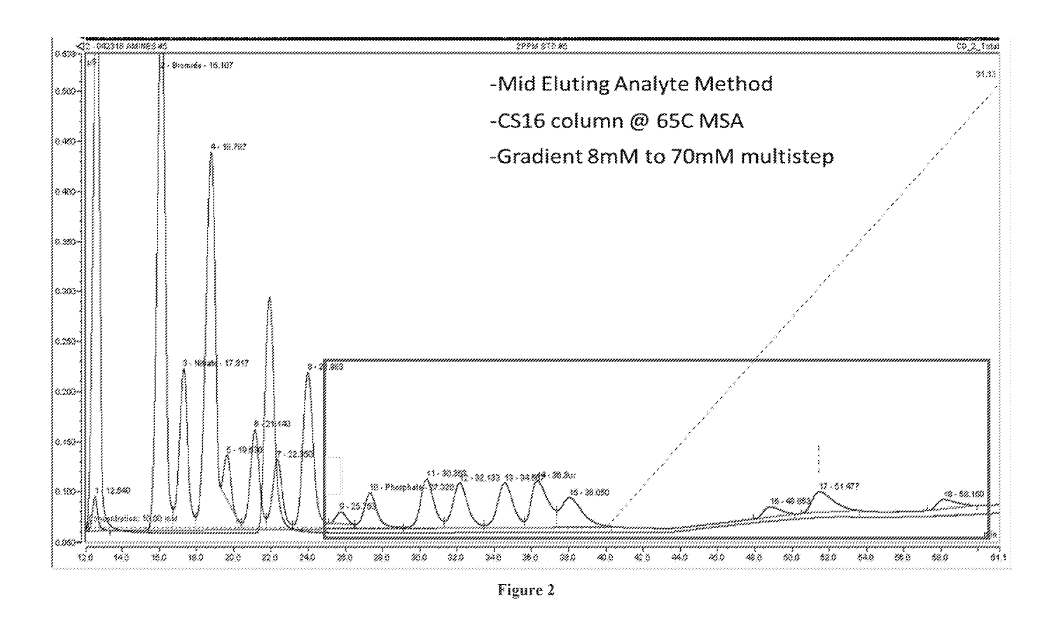

The result of the analysis is presented in FIG. 2.

### Third Chromatography Step—Chromatography Conditions:

### Dionex ICS3000

IC column: CS19 plus CG19 guard column

Column Temperature: 39° C.

Injection Volume: 10 uL

Eluent: Methanesulfonic Acid (MSA)

Eluent Flow Rate: 0.25 ml/min

Separation Parameters: 10 mM MSA isocratic to 50 min

Detector: Conductivity

Detector temperature: 20° C.

Run time: 45 min.

### Results:

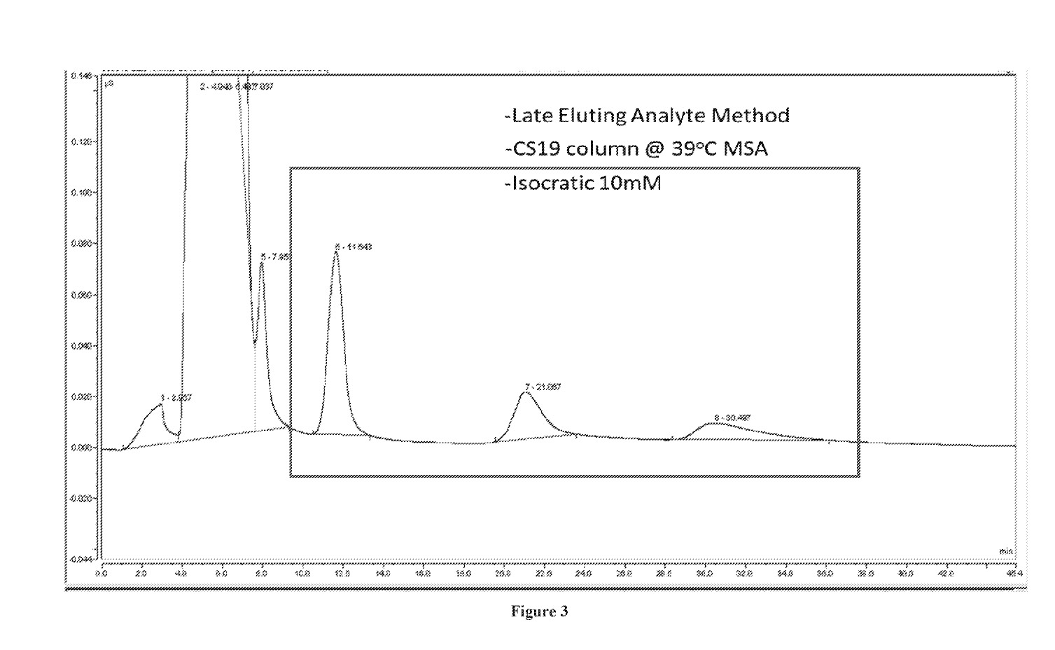

The result of the analysis is presented in FIG. 3.

### Example 2: Analysis of an Amine Mixture

### Material:

Sample containing a mixture of amines

### First Chromatography Step—Chromatography Conditions:

### Metrohm 940 Professional IC Vario

IC column: Metrohm™ Metrosep C6
IC column dimension: 250 mm long×4 mm inner diameter

### Injection Volume: 10 μl

First Eluent: 3.4 mM oxalic acid and 0.25% acetonitrile. The first eluent was prepared by adding oxalic acid dihydrate (0.857 g+/−0.001 g) into a 2 L glass container followed by addition of acetonitrile (5 ml). The mixture was diluted to 2 L with deionized or distilled water. The resulting mixture was mixed for ˜10 minutes to ensure dissolution of oxalic acid and degassing of the acetonitrile.

Second eluent: 50 mM oxalic acid 10% acetonitrile. The second eluent was prepared by adding oxalic acid dihydrate (12.6 g+/−0.1 g) into a 2 L glass container followed by addition of acetonitrile (200 ml). The mixture was dilute to 2 L by adding deionized or distilled water. The resulting mixture was mixed for ˜10 minutes to ensure dissolution of oxalic acid and degassing of the acetonitrile.

Eluent Flow Rate: 1.0 ml/min

Column Temperature: 25° C.

Separation Parameters: first eluent isocratic to 85 min; switch to second eluent; second eluent isocratic to 90 min; switch to first eluent; first eluent isocratic to 120 min.

Detector: Conductivity

Detector temperature: 40° C.

Run time: 120 min.

### Results:

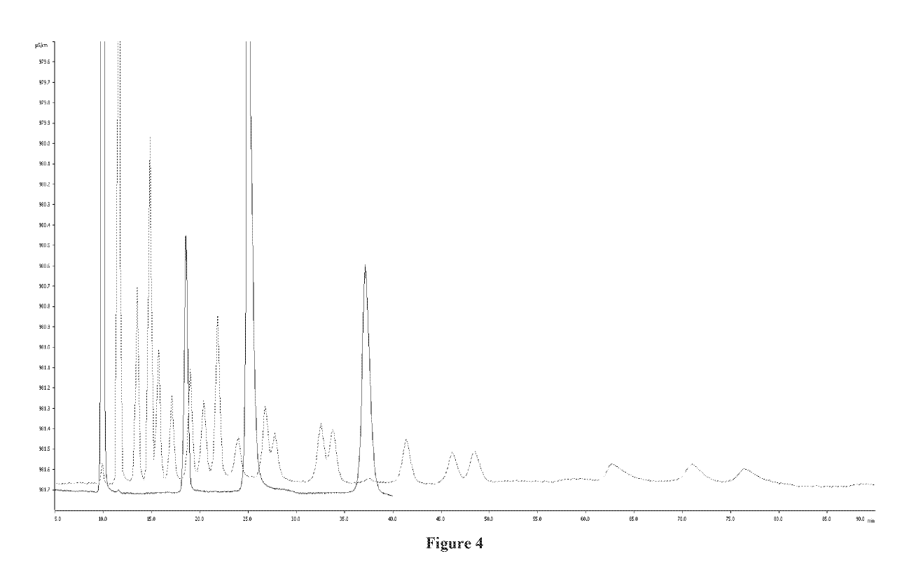

The result of the analysis is presented in FIG. 4.

### Second Chromatography Step—Chromatography Conditions:

### Metrohm 940 Professional IC Vario

IC column: Metrohm™ Metrosep C6
IC column dimension: 250 mm long×4 mm inner diameter

### Column Temperature: 25° C.

Injection Volume: 10 uL

Eluent: 10 mM oxalic acid and 2.5% acetonitrile. The eluent was prepared by adding oxalic acid dihydrate (2.52 g+/−0.01 g) into a 2 L glass container followed by addition of acetonitrile (50 ml). The mixture was diluted to 2 L with deionized or distilled water. The resulting mixture was shaken for ˜10 minutes to ensure dissolution of oxalic acid and degassing of the acetonitrile.

Eluent Flow Rate: 1.0 ml/min

Separation Parameters: isocratic to 53 min.

Detector: Conductivity

Detector temperature: 53° C.

Run time: 52 minutes.

### Results:

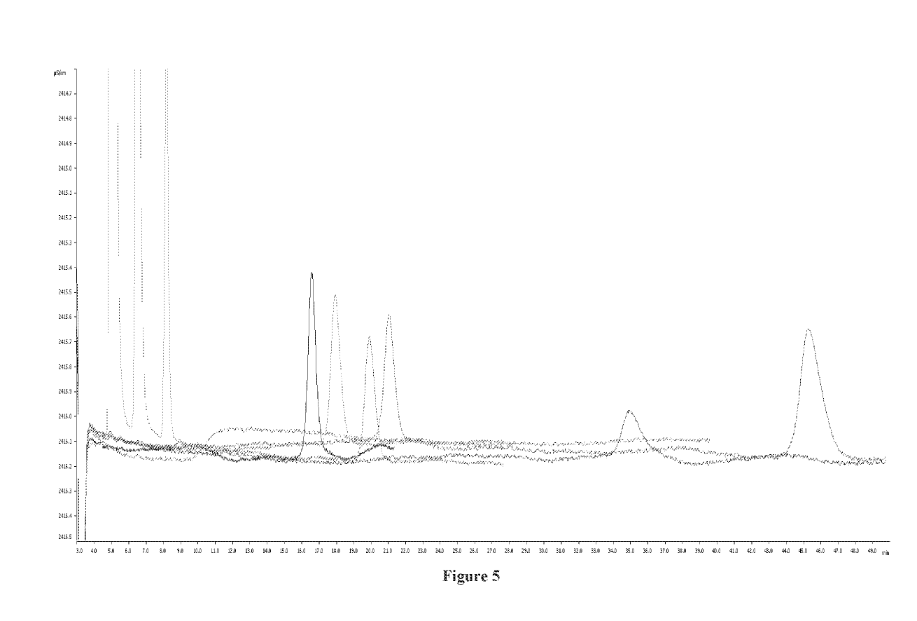

The result of the analysis is presented in FIG. 5.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative materials and method steps disclosed herein are specifically described, other combinations of the materials and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

What is claimed is:

1. A method for separating and/or monitoring a plurality of amines in a mixture, the method comprising:
(a) a first chromatography step comprising
i. introducing a sample of the mixture onto a first ion-exchange chromatography matrix; and
ii. eluting at least one first eluate from the first ion-exchange chromatography matrix under a first multistep gradient eluting condition, using a first gradient eluent at a first elution temperature; and
(b) a second chromatography step comprising
i. introducing a sample of the mixture onto a second ion-exchange chromatography matrix; and
ii. eluting at least one second eluate from the second ion-exchange chromatography matrix under a first isocratic condition, using a first isocratic eluent at a second elution temperature;
wherein the mixture comprises at least five amines and the method is performed in less than about 3 hours.

2. The method of claim 1, wherein the first gradient eluent and the first isocratic eluent comprise an acid, wherein the acid is independently selected from citric acid, glycine, 2(N-morpholino)ethanesulfonic acid, methanesulfonic acid, acetic acid, formic acid, phosphoric acid, hydrochloric acid, nitric acid, sulfuric acid, phosphoric acid, oxalic acid, tartaric acid, benzoic acid, phthalic acid, and combinations thereof.

3. The method of claim 2, wherein the first gradient eluent and the first isocratic eluent comprise oxalic acid.

4. The method of claim 2, wherein the first gradient eluent and the first isocratic eluent further comprise acetonitrile.

5. The method of claim 1, wherein the first multistep gradient eluting condition comprises an acid concentration gradient of from about 2 mM to about 100 mM and acetonitrile in an amount of from about 0.05% to about 25% by volume, based on the total volume of the first gradient eluent.

6. The method of claim 1, wherein the first multistep gradient condition includes
a first step, wherein the first gradient eluent comprises an acid at a concentration of from about 2 mM to about 5 mM and acetonitrile in an amount of from about 0.05% to about 5% by volume, based on the total volume of the first gradient eluent;
a second step, wherein the first gradient eluent comprises an acid at a concentration of from about 25 mM to about 100 mM and acetonitrile in an amount of from about 5% to about 20% by volume, based on the total volume of the first gradient eluent; and
a third step, wherein the first gradient eluent comprises an acid at a concentration of from about 2 mM to about 5 mM and acetonitrile in an amount of from about 0.05% to about 5% by volume, based on the volume of the first gradient eluent.

7. The method of claim 1, wherein the first elution temperature is from about 15° C. to about 40° C.

8. The method of claim 1, wherein the first isocratic eluent has a concentration of acid from about 5 mM to about 20 mM and acetonitrile in an amount of from about 0.5% to about 5% by volume, based on the total volume of the first isocratic eluent.

9. The method of claim 8, wherein acetonitrile is present in an amount of from about 2% to about 4% by volume, based on the total volume of the first isocratic eluent.

10. The method of claim 1, wherein the second elution temperature is from about 15° C. to about 40° C.

11. The method of claim 1, wherein steps (a) and (b) are performed simultaneously in a dual channel apparatus.

12. The method of claim 1, wherein the first ion-exchange chromatography matrix and the second ion-exchange chromatography matrix include a carboxylic acid resin.

13. The method of claim 1, wherein the mixture is an industrial fluid.

14. The method of claim 13, wherein the industrial fluid is selected from the group consisting of a refinery fluid, a production fluid, cooling water, process water, a drilling fluid, a completion fluid, a production fluid, crude oil, a feed stream to a desalting unit, an outflow from a desalting unit, a refinery heat transfer fluid, a gas scrubber fluid, a refinery unit feed stream, a refinery intermediate stream, a finished product stream, and a combination thereof.

15. The method of claim 1, wherein the mixture comprises from about 5 to about 25 amines.

16. The method of claim 15, wherein the mixture comprises at least 20 amines.

17. The method of claim 1, further comprising identifying and/or quantifying the separated amines using an apparatus selected from a mass spectrometer, a nuclear magnetic resonance spectrometer, a surface enhanced Raman spectrometer, a ultra-violet spectrophotometer, a fluorimeter, a conductivity detector, and combinations thereof wherein the method is performed in less than about 24 hours.

18. The method of claim 17, wherein the method is directed to the efficient operation of a refinery.

19. A method for separating and/or monitoring a plurality of amines in a mixture, the method comprising:
(a) a first chromatography step comprising
i. introducing a sample of the mixture onto a first ion-exchange chromatography matrix;
ii. eluting at least one first eluate from the first ion-exchange chromatography matrix under a first gradient eluting condition, using a first gradient eluent at a first elution temperature;
(b) a second chromatography step comprising
i. introducing a sample of the mixture onto a second ion-exchange chromatography matrix;
ii. eluting at least one second eluate from the second ion-exchange chromatography matrix under a second gradient eluting condition, using a second gradient eluent at a second elution temperature; and
(c) a third chromatography step comprising
i. introducing a sample of the mixture onto a third ion-exchange chromatography matrix;
ii. eluting at least one third eluate from the third ion-exchange
chromatography matrix under a second isocratic eluting condition, using a second isocratic eluent at a third elution temperature; and
wherein the mixture comprises at least five amines and the method is performed in less than about 24 hours.

20. The method of claim 19, wherein the first gradient eluent, the second gradient eluent, and the second isocratic eluent are independently selected from citric acid, glycine, 2(N-morpholino)ethanesulfonic acid, methanesulfonic acid, acetic acid, formic acid, phosphoric acid, hydrochloric acid, nitric acid, sulfuric acid, phosphoric acid, oxalic acid, tartaric acid, benzoic acid, phthalic acid, and combinations thereof.

21. The method of claim 20, wherein the first gradient eluting condition includes an acid concentration gradient of between about 1 mM to about 100 mM.

22. The method of claim 19, wherein the first gradient eluting condition includes a first multistep gradient, wherein the first multistep gradient includes
a first step, wherein the first gradient eluent comprises an acid concentration of from about 1 mM to about 10 mM;
a second step, wherein the first gradient eluent comprises an acid concentration of from about 50 mM to about 100 mM;
a third step, wherein the first gradient eluent comprises an acid concentration of from about 1 mM to about 10 mM.

23. The method of claim 19, wherein the first elution temperature is from about 35° C. to about 45° C.

24. The method of claim 20, wherein the second gradient eluting condition includes an acid concentration gradient of between about 1 mM to about 100 mM.

25. The method of claim 19, wherein the second gradient eluting condition includes a second multistep gradient, wherein the second multistep gradient includes
a first step, wherein the first gradient eluent comprises an acid concentration of from about 3 mM to about 15 mM;
a second step, wherein the first gradient eluent comprises an acid concentration of from about 50 mM to about 100 mM;
a third step, wherein the first gradient eluent comprises an acid concentration of from about 3 mM to about 10 mM.

26. The method of claim 19, wherein the second elution temperature is from about 55° C. to about 70° C.

27. The method of claim 20, wherein the second isocratic eluent comprises an acid at a concentration of from about 5 mM to about 20 mM.

28. The method of claim 19, wherein the third elution temperature is from about 35° C. to about 45° C.

29. The method of claim 19, wherein steps (a) and (b) are performed simultaneously in a dual channel apparatus.

30. A method for separating and/or monitoring a plurality of amines in a mixture, the method comprising:
(a) a first chromatography step comprising
i. introducing a sample of the mixture onto a first ion-exchange chromatography matrix;
ii. eluting at least one first eluate from the first ion-exchange chromatography matrix under a first multistep gradient eluting condition, using a first gradient eluent at a first elution temperature of from about 35° C. to about 45° C.;
(b) a second chromatography step comprising
i. introducing a sample of the mixture onto a second ion-exchange chromatography matrix;
ii. eluting at least one second eluate from the second ion-exchange chromatography matrix under a second multistep gradient eluting condition, using a second gradient eluent at a second elution temperature of from about 55° C. to about 70° C.;
(c) optionally, a third chromatography step comprising
i. introducing a sample of the mixture onto a third ion-exchange chromatography matrix;
ii. eluting at least one third eluate from the third ion-exchange chromatography matrix under a second isocratic eluting condition, using a second isocratic eluent at a third elution temperature of from about 35° C. to about 45° C.;
wherein the mixture comprises at least five amines and the method is performed in less than about 24 hours.
